(19) United States
(12) Patent Application Publication
(10) Pub. No.: US 2021/0187013 A1
(43) Pub. Date: Jun. 24, 2021

(54) Methods of Treating and Preventing Cancer by Disrupting the Binding of Copper in the Map Kinase Pathway

(71) Applicant: DUKE UNIVERSITY, Durham, NC (US)

(72) Inventors: Chris M. Counter, Durham, NC (US); Donita C. Brady, Durham, NC (US); Michelle L. Turski, San Francisco, CA (US); Dennis J. Thiele, Chapel Hill, NC (US)

(73) Assignee: DUKE UNIVERSITY, Durham, NC (US)

(21) Appl. No.: 17/144,967

(22) Filed: Jan. 8, 2021

Related U.S. Application Data
(63) Continuation of application No. 14/371,871, filed on Jul. 11, 2014, now abandoned.
(60) Provisional application No. 61/585,401, filed on Jan. 11, 2012.
(60) Provisional application No. 61/702,406, filed on Sep. 18, 2012.

(51) Int. Cl. A61K 33/24; A61K 45/06; A61K 33/30; A61K 31/095; A61K 31/519; A61K 31/275; A61K 31/4523; A61K 31/132; A61K 31/4745; A61K 31/198; A61K 31/18; A61K 31/416

(52) U.S. Cl. CPC A61K 33/24; A61K 45/06; A61K 33/30; A61K 31/095; A61K 31/519; A61K 31/416; A61K 31/4523; A61K 31/132; A61K 31/4745; A61K 31/198; A61K 31/18; A61K 31/275

(57) ABSTRACT

The present disclosure provides methods of treating and/or preventing cancer in a subject comprising administering to the subject a copper-reduced diet by itself or as a supplement along with a regular diet to create a copper-reduced melieu, maintain a reduced-copper melieu, or both, thereby treating and/or preventing the development of the cancer. Methods also comprise further adding a copper chelator, MEK inhibitor, or combinations thereof.

### CROSS REFERENCE TO RELATED APPLICATIONS

This application is a continuation of U.S. patent application Ser. No. 14/371,871, filed Jul. 11, 2014, which is a U.S. national phase of International Application No. PCT/US2013/021194, filed Jan. 11, 2013, which claims the benefit of priority to U.S. Provisional Patent Application No. 61/585,401, filed Jan. 11, 2012 and U.S. Provisional Patent Application No. 61/702,406, filed Sep. 18, 2012, the contents of each of which are incorporated herein by references in their entirety.

### FEDERAL FUNDING LEGEND

This invention was made with Government Support under Federal Grant Nos. R01 CA094184, DK074192 and 5R21CA172104-02 awarded by the NIH. The Government has certain rights to this invention.

### BACKGROUND

Activating V-Raf murine sarcoma viral oncogene homolog B 1 (BRAF) mutations are prevalent in numerous types of cancers, including 50-70% of melanomas, 15% of colorectal and ovarian cancers, and 36-69% of papillary thyroid carcinomas (reviewed in Davies, H. et al., (2002) Nature, 417:949-954; and Namba, H. et al. (2003) J Clin. Endocr. Metab., 88:4393-97). Activating BRAF mutations have also been identified in up to 82% of benign melanocytic tumors (nevi) (Pollock, P. M. et al. (2003) Nature Genet. 33:19-20). The most common activating BRAF mutation is a glutamic acid to valine substitution at position 600 (V600E; formerly identified as V599E). This mutation produces a highly active kinase that stimulates constitutive extracellular signal-regulated protein kinase (ERK) signaling. Expression of BRAF^{V600E} has been shown to induce senescence in cultured human fibroblasts (Zhu, J. et al. (1998) Genes Dev., 12:2997-3007) and human melanocytes (Michaloglou, C. et al. (2005) Nature 436:720-724) and in vivo in preneoplastic nevi (Michaloglou, C. et al. (2005) Nature 436:720-724).

Copper is a key nutrient for biological processes including mitochondrial respiration and free radical detoxification. Ctr1 is a copper transporter located on the cell membrane. This ATP independent transporter has a high affinity for copper and allows sufficient amounts of copper to enter the cell for normal metabolic function. Ctr1 has recently been identified as one of approximately 300 genes that when knocked down in S2 insect cells reduced phosphorylation of Erk, suggesting that copper transport is required for MAPK signaling (see, e.g., Turski, M. L. et al. (2012), Mol. Cell. Biol., 32:1284-1295). Moreover, recent studies have also shown that activation of Erk1/2 by oncogenic Ras^{G12V} and BRaf^{V600E} was greatly reduced in mouse embryonic fibroblasts (MEFs) homozygous null for the Ctr1 gene, and that the defect lies at the level of Mek1/2 (see, e.g., Turski, M. L. et. al. (2012), supra). Indeed, Mek1 binds directly to copper and requires copper for kinase activity in vitro, suggesting that copper is a co-factor for Mek1/2 activity (see, e.g., Turski, M. L. et. al. (2012), supra).

### SUMMARY OF THE INVENTION

The present disclosure is based, in part, on the surprising discovery that copper is critical for Mek1/2 to promote oncogenic BRaf-dependent tumor growth.

One aspect of the present disclosure provides a method of treating a cancer in a subject comprising, consisting of, or consisting essentially of administering to the subject a copper-reduced diet by itself or as a supplement along with a regular diet to create a copper-reduced melieu, maintain a reduced-copper melieu, or both, thereby treating the cancer.

Another aspect of the present disclosure provides a method of preventing a cancer from developing in a subject comprising, consisting of, or consisting essentially of administering to the subject a copper-reduced diet by itself or as a supplement along with a regular diet to create a copper-reduced melieu, maintain a reduced-copper melieu, or both, thereby preventing the cancer from developing.

Yet another aspect of the present disclosure provides methods of treating or preventing melanoma in a subject comprising, consisting of, or consisting essentially of administering to the subject a copper-reduced diet by itself or as a supplement along with a regular diet to create a copper-reduced melieu, maintain a reduced-copper melieu, or both, thereby treating the cancer.

Yet another aspect of the present disclosure provides methods of treating cancer and/or preventing a cancer from developing in a subject comprising, consisting of, or consisting essentially of administering to the subject a MEK inhibitor, the inhibitor being capable of blocking the binding of copper to MEK1 and/or MEK2.

In some embodiments, the cancer is characterized by increased Ras-BRaf-Mek-Erk signaling, is dependent for growth and/or survival upon the Ras-BRaf-Mek-Erk signaling pathway, and/or expresses an activated or oncogenic BRaf, Ras or Mek. In certain embodiments, the activated or oncogenic BRaf comprises BRaf^{V600E}. In other embodiments, the activated or oncogenic Ras comprises Ras^{G12V}

In yet other embodiments, the cancer is selected from the group consisting of carcinoma, breast cancer, ovarian cancer, pancreatic cancer, colon cancer, colorectal cancer, colon cancer, papillary thyroid carcinoma, melanoma, bladder, testicular, head and neck, cervical cancer, lung cancer, Wilms' tumor, brain tumor, neuroblastoma, retinoblastoma, mesothelioma, esophageal cancer or hairy cell leukemia. In certain embodiments, the cancer comprises melanoma.

In other embodiments, the methods further comprise, consist of, or consist essentially of administering to the subject a copper chelator.

In certain embodiments, the copper chelator is selected from the group consisting of penicillamine, bathocuprione sulfonate, sodium diethyldithiocarbamate, trientine hydrocholoride, dimercaprol, ammonium tetrathiomolybdate (TM), zinc acetate and combinations thereof.

In other embodiments, the methods further comprise, consist of, or consist essentially of administering to the subject a chemotherapeutic and/or anti-cancer agent. In some embodiments, the method comprises administering an anti-cancer agent. In other embodiments, the anti-cancer agent is a MEK inhibitor. In some embodiments, the MEK inhibitor is capable of blocking the binding of copper to MEK. In certain embodiments, the MEK inhibitor is selected from the group consisting of butanedinitrile, GSK1120212, XL518, selumetinib, bis[amino[2-aminophenyl)thio]methylene]-(9C1), (N-(3-chloro-4-fluorophenyl)-7-methoxy-6-(3-morpholin-4-ylpropoxy)quinazolin-4-amine), (N-[(2R)-2,3-dihydroxypropoxy]-3,4-difluoro-2-[(2-fluoro-4-iodophenyl)amino]-benzamide), (2′-amino-3′-methoxyflavone), (1,4-diamino-2,3-dicyano-1,4-bis(aminophenylthio)butadiene), (6-(4-Bromo-2-chloro-phenylamino)-7-fluoro-3-methyl-3H-benzoimidazole-5-carboxylic acid (2-hydroxyethoxy)-amide, [2-(2-fluoro-4-iodophenylamino)-N-(2-hydroxyethoxy)-1,5-dimethyl-6-oxo-1,6-dihydropyridine-3-carboxamide, (2-(2-Chloro-4-iodophenylamino)-N-cyclopropylmethoxy-3,4-difluoro-benzamide), N-[(R)-2,3-Dihydroxy-propoxy]-3,4-difluoro-2-(2-fluoro-4-iodophenylamino)-benzamide, U0126 and combinations thereof.

Another aspect of the present disclosure provides for all that is disclosed and illustrated herein.

### BRIEF DESCRIPTION OF THE DRAWINGS

The foregoing aspects and other features of the invention are explained in the following description, taken in connection with the accompanying drawings, wherein:

FIGS. 1A-1C are images and graphs showing the large-fly phenotype resulting from the knockdown of Ctr1A in the prothoracic gland. FIG. 1A shows reduced plasma membrane staining of Ctr1A in the prothoracic gland in Ctr1A knockdown cells as detected through indirect immunofluorescence assay. FIG. 1B shows the relative size of an adult female Drosophila fly carrying the prothoracic gland driver, (Phm-Gal4) an adult female fly with knockdown of Ctr1A in the prothoracic gland (Phm-Gal4: UAS-Ctr1A^{RNAi}). FIG. 1C shows quantitative measurements of pupae based on the sex of the fly and genotype.

FIGS. 2A-2B are images showing the effect of Ctr1A knockdown on the constitutively active Ras phenotypes in both the fly eye and wing. FIG. 2A shows bright-field images of adult Drosophila wings. Expression of UAS-Ras^{V12} using an apterous-Gal4 (ap-Gal4) driver, which drives expression in the dorsal compartment of the wing, is lethal, while expression of both the UAS-Ras^{V12} and UAS-Ctr1A^{RNAi} transgenes yields viable adult flies with normal wings. FIG. 2B shows an SEM image of adult female Drosophila eyes, with the genotype shown above each image. The rough-eye phenotype after Ctr1 and MAPK activation is shown. The rough-eye phenotype in Ey-Gal4: UAS-Ras^{G14V} is rescued in Ey-Gal4: UAS-Ras^{G14V}, UAS-Ct1^{RNA1} flies.

FIGS. 3A-3C are immunoblots showing copper chelation or competition for Ctr1A-mediated Cu⁺ transport compromises Ras/MAPK signaling in Drosophila S2 cells by analyzing total protein extracts. FIG. 3A shows levels of total Erk and phosphor-Erk (P-Erk) in cells that were not pretreated (−) or pretreated with the Cu⁺-specific membrane-impermeant chelator BCS or with insulin from 0 to 15 minutes. FIG. 3B shows the same experiment as panel 3a using the membrane-impermeant Fe²⁺-specific chelator BPS. FIG. 3C shows the same experiment as panel 3a using no pre-treatment or pretreatment with silver (Ag).

FIGS. 4A-4B are immunoblots showing that Ctr1 function in Ras/MAPK signaling is dependent on Cu⁺ transport activity. FIG. 4A shows phospho-Erk levels over time in Ctr1^{+/+} and Ctr1^{-/-} MEFs that were treated with insulin. FIG. 4B shows insulin-stimulated Ras/MAPK activity in the phosphorylation of Erk in Ctr1^{-/-} cells stably expressing either wild-type human Ctr1 (Ctr1) or a transport-defective mutant form of human Ctr1 (Ctr1^{M150A}).

FIG. 5 is an immunoblot showing the phosphorylated and total levels of B-Raf, Mek1/2, Erk1/2, and Akt1 from Ctr1^{+/+} and Ctr1^{-/-} cells that were serum starved for 16 hours and subsequently stimulated with FGF at minutes 0, 5, and 10.

FIGS. 6A-6C are immunblots showing Mek1 affinity purified by Cu-chelated resins. FIG. 6A shows the levels of Mek1, GADPH, and Erk1/2 as assayed from input proteins, GSH resin affinity-purified proteins, and Cu-charged GSH resin-purified proteins. FIG. 6B shows an immunoblotting assay of Mek1 and KSR1 scaffold proteins by incubating pentadentate-chelated beads complexed with no metal, zinc, or Cu with Ctr1^{+/+} cell lysate. FIG. 6C shows the SDS-PGAE and immunoblotting assay of purified recombinant rat Mek1 that was added to uncharged pentadentate beads or charged with zinc or copper, and then affinity purified.

FIGS. 7A-7D are graphs and a table showing recombinant Mek1 metal-binding characteristics. FIG. 7A shows the Cu/Mek1 binding ratio from dialysis experiments and competition experiments under the indicated equilibrium conditions. FIG. 7B shows the saturation of binding equilibrium dialysis with increasing CuCl₂ concentrations in the dialysate using an independent set of purified rat Mek1. FIG. 7C shows the Cu²⁺ dissociation constant, K_{D}, of Mek1 using the probe PAR showing overall spectral changes of the Cu-PAR complex on Mek1 titration. The inset shows the decrease at 500 nm relative to Mek1 additions for [Cu-PAR]ₜₒₜₐₗ of 3.9 μM and a [PAR]ₜₒₜₐₗ of 9.3 μM. FIG. 7D shows apparent K_{D}s at pH 7.4 derived from competition titration using Cu²⁺-PAR.

FIG. 8 is an immunoblot showing that copper is a co-factor of Mek. An in vitro kinase assay reveals increasing CuSO₄ elevates recombinant Erk1 phosphorylation by recombinant Mek1.

FIGS. 9A-9D are Western blots and a graph showing that Mek1 kinase activity and association with Erk are stimulated by Cu. FIG. 9A shows a Western blot with Erk1/2 phosphospecific antibody of recombinant, GST-tagged human kinase-dead Erk2 and recombinant GST-tagged human Mek1 incubated with increasing amounts of CuSO₄, with or without TTM or Mek1 inhibitor. FIG. 9B shows a Western blot with MBP phosphospecific antibody of recombinant GST-hErk2 and recombinant MBP incubated with increasing amounts of CuSO4. FIG. 9C shows coimmunoprecipitation of Mek1 and Erk1/2 in Ctr1^{+/+} and Ctr1^{-/-} MEFs as assessed by Western blotting with Mek1 and Erk1/2 antibodies. RalB immunoprecipitation was used as a negative control. CCS protein levels of whole-cell extract was used to assess Cu deficiency. FIG. 9D is a graph showing that Mek1 kinase activity and association with Erk are stimulated by Cu.

FIG. 10 is an immunoblot showing that the loss of Ctr1 reduces Erk1/2 activation. Immunoblot analyses reveal Erk1/2 phosphorylation is reduced in CTR1^{-/-} compared to CTR1^{+/+} MEFs transformed with SV40 and Braf^{V600E} Ras^{G12V}.

FIG. 11 is an immunoblot showing Ras/MAPK signaling of heart lysates from Ctr1 wild-type animals (Ctr1^{flox/flox}) and mutant mice with cardiac-tissue-specific ablation of Ctr1 expression (Ctr1^{hrt/hrt}).

FIGS. 12A-12C are graphs showing that copper is required for BRaf^{V600E}-driven tumorigenesis. FIG. 12A shows percent (%) survival (time to reach maximum tumor mass) versus time of mice injected with BRaf^{V600E}+SV40 transformed Ctr1^{+/+} (black line) or Ctr1^{-/-} (red line) MEFs. FIG. 12B shows tumor volume versus time of mice injected with BRafV600E+SV40 transformed MEFs: expressing a scramble (▪) or Mek1 shRNA with no transgene (♦) or 187/8A ( [symbol] ) or 230/9A ( [symbol] ) copper-binding Mek1 mutants. FIG. 12C shows tumor volume versus time of mice untreated (●) or treated with 2 mg/day oral TM ( [symbol] ).

### DETAILED DESCRIPTION OF THE INVENTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

Unless otherwise defined, all technical terms used herein have the same meaning as commonly understood by one of ordinary skill in the art to which this disclosure belongs.

As used herein, the term “subject” is intended to include human and non-human animals. Exemplary human subjects include a human patient having a disorder, e.g., a disorder described herein, or a normal subject. The term “non-human animals” includes all vertebrates, e.g., non-mammals (such as chickens, amphibians, reptiles) and mammals, such as non-human primates, domesticated and/or agriculturally useful animals (such as sheep, dogs, cats, cows, pigs, etc.), and rodents (such as mice, rats, hamsters, guinea pigs, etc.).

“Effective amount,” as used herein, refers to (i) the amount of a desired element in a diet, e.g., copper, or (ii) a dosage of the compounds or compositions effective for eliciting a desired effect. This term as used herein may also refer to an amount effective at bringing about a desired in vivo effect in an animal, mammal, or human, such as reducing proliferation of a cancer cell.

“Reducing proliferation of a cell,” as used herein, refers to reducing, inhibiting, or preventing the survival, growth, or differentiation of a cell, including killing a cell. A cell can be derived from any organism or tissue type and includes, for example, a cancer cell (e.g., neoplastic cells, tumor cells, and the like).

As used herein, the term “treat” or “treating” a subject having a disorder refers to administering a regimen to the subject, e.g., the administration of a combination of a copper chelator and a platinum-based therapeutic, such that at least one symptom of the disorder is cured, healed, alleviated, relieved, altered, remedied, ameliorated, or improved. Treating includes administering an amount effective to alleviate, relieve, alter, remedy, ameliorate, improve or affect the disorder or the symptoms of the disorder. The treatment may inhibit deterioration or worsening of a symptom of a disorder.

As used herein the term “prevention” means generally the prevention of the establishment of a cancer. Prevention may be primary, secondary or tertiary. For example, primary prevention refers to the prevention of the establishment of the disease. Secondary prevention refers to intervention in subjects who are at high risk for the development of a cancer but have not yet developed the disease. These subjects may or may not have exhibited some physiological symptoms. These individuals may also have a family history of cancer. Tertiary prevention refers to preventing the worsening of the cancer and reducing the symptoms experienced by the subjects.

“Pharmaceutically acceptable,” as used herein, pertains to compounds, materials, compositions, and/or dosage forms which are, within the scope of sound medical judgment, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

One aspect of the present disclosure provides methods of treating and/or preventing a cancer in a subject comprising, consisting of, or consisting essentially of administering to the subject a copper-reduced diet by itself or as a supplement along with a regular diet to create a copper-reduced melieu, maintain a reduced-copper melieu, or both, thereby treating and/or preventing the cancer.

Copper is provided primarily through diet. A copper-reduced diet comprises of foods that are low or null in copper content. Such foods include oysters and other shellfish, whole grains, beans, nuts, potatoes, organ meats (e.g., liver, kidney), dark, leafy greens, dried fruits, cocoa, black pepper, and yeast.

The term “cancer” refers to cells having the capacity for autonomous growth. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. Also included are malignancies of the various organ systems, such as respiratory, cardiovascular, renal, reproductive, hematological, neurological, hepatic, gastrointestinal, and endocrine systems; as well as adenocarcinomas which include malignancies such as most colon cancers, renal-cell carcinoma, prostate cancer and/or testicular tumors, non-small cell carcinoma of the lung, cancer of the small intestine, and cancer of the esophagus. Cancer that is “naturally arising” includes any cancer that is not experimentally induced by implantation of cancer cells into a subject, and includes, for example, spontaneously arising cancer, cancer caused by exposure of a patient to a carcinogen(s), cancer resulting from insertion of a transgenic oncogene or knockout of a tumor suppressor gene, and cancer caused by infections, e.g., viral infections. The term “carcinoma” is art recognized and refers to malignancies of epithelial or endocrine tissues. Examples of cancers that are within the scope of the present disclosure include, but are not limited to, carcinoma, breast cancer, ovarian cancer, pancreatic cancer, colon cancer, colorectal cancer, colon cancer, papillary thyroid carcinoma, melanoma, bladder, testicular, head and neck, cervical cancer, lung cancer, Wilms' tumor, brain tumor, neuroblastoma, retinoblastoma, mesothelioma, esophageal cancer or hairy cell leukemia. In particular embodiments, the cancer is melanoma. In some embodiments, the cancer is characterized by increased Ras-BRaf-Mek-Erk signaling, is dependent for growth and/or survival upon the Ras-BRaf-Mek-Erk signaling pathway, and/or expresses an activated or oncogenic BRaf, Ras or Mek. Any mutations in BRaf, Ras and/or Mek are within the scope of the present disclosure. In certain embodiments, the activated or oncogenic BRaf comprises BRaf^{V600E}. In other embodiments, the activated or oncogenic Ras comprises Ras^{G12V}.

In some embodiments, the methods of the present disclosure further comprise administering to the subject a compound(s) that also help prevent the uptake of copper by the subject. Such compounds include, but are not limited to, copper chelators.

As sued herein, the term “administration” or “administering,” as used herein, refers to providing, contacting, and/or delivery of a diet, compound or compounds by any appropriate route to achieve the desired effect. For example, administering a copper-reduced diet may comprise the design, preparation, and/or delivery of food low in copper content to the subject. In certain embodiments, the term “administration” may also include the delivery of a compound, such as a copper chelator. These compounds may be administered to a subject in numerous ways including, but not limited to, oral, sublingual, parenteral (e.g., intravenous, subcutaneous, intracutaneous, intramuscular, intraarticular, intraarterial, intrasynovial, intrasternal, intrathecal, intralesional or intracranial injection), transdermal, topical, buccal, rectal, vaginal, nasal, ophthalmic, via inhalation, and implants.

Copper chelators are compounds capable of creating a copper deficient environment, e.g., around a cancer cell or a tumor. Mutations in copper transporters such as in Wilson disease (export pump ATP7B) result in copper accumulation in the tissues and copper toxicity in several major organ systems (Schilsky, M. L. (2009) Biochimie 91(10): 1278-81). Copper chelation is necessary in subjects with these diseases to reduce copper levels and toxicity. Accordingly, several copper chelators are approved for use in these subjects, and may be used in the methods described herein to reduce copper levels.

Embodiments of the methods described herein provide for a copper chelator that binds copper in the Cu(I) or Cu(II) oxidation state. Some embodiments provide for a copper chelator having a higher binding affinity for Cu(I) relative to Cu(II). Some embodiments provide for a copper chelator having a higher binding affinity for Cu(II) relative to Cu(I). Copper chelators may include without limitation: penicillamine (Cuprimine™, Depen™), trientine hydrochloride (also known as triethylenetetramine hydrochloride, or Syprine™), dimercaprol, diethyldithiocarbamate (e.g., sodium diethyldithiocarbamate), bathocuproine sulfonate, and tetrathiomolybdate (e.g., ammonium tetrathiomolybdate (TM)). In some embodiments, the copper chelator is not tetrathiomolybdate.

Tetrathiomolybdate, such as ammonium tetrathiomolybdate, may serve to chelate copper and may also compete with copper for intestinal absorption. Other compounds used to control copper levels in patients with Wilson disease include zinc salts, such as zinc acetate (Galzin™), which also compete with copper for intestinal absorption. Zinc may also induce production of metallothionein, a protein that binds copper and prevents its transfer into the bloodstream. Accordingly, tetrathiomolybdate and/or zinc may also be used to reduce copper absorption in the methods described herein.

It is also within the scope of the present disclosure that the methods comprise the co-administration of a copper reducing diet together with a copper chelator. Administered “in combination,” as used herein, means that two (or more) different treatments are delivered to the subject during the course of the subject's affliction with the disorder, e.g., the two or more treatments (e.g., a copper reduced diet and administration of one or more copper chelators) are delivered after the subject has been diagnosed with the disorder and before the disorder has been cured or eliminated or treatment has ceased for other reasons. In some embodiments, the delivery of one treatment (e.g., a copper reduced diet) is still occurring when the delivery of the second begins (e.g., administration of one or more copper chelators), so that there is overlap in terms of administration. This is sometimes referred to herein as “simultaneous” or “concurrent delivery.” In other embodiments, the delivery of one treatment ends (e.g., copper reduced diet) before the delivery of the other treatment begins (e.g., administration of a copper chelator). In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In some embodiments, the copper reduced diet and one or more copper chelator are administered in combination with other therapeutic treatment modalities, including surgery, radiation, cryosurgery, and/or thermotherapy. Such combination therapies may advantageously utilize lower dosages of the administered agent and/or other chemotherapeutic agent, thus avoiding possible toxicities or complications associated with the various therapies. The phrase “radiation” includes, but is not limited to, external-beam therapy which involves three dimensional, conformal radiation therapy where the field of radiation is designed to conform to the volume of tissue treated; interstitial-radiation therapy where seeds of radioactive compounds are implanted using ultrasound guidance; and a combination of external-beam therapy and interstitial-radiation therapy.

In some embodiments, the copper reduced diet and one or more copper chelator are administered with at least one additional therapeutic agent, such as a chemotherapeutic and/or anti-cancer agent. Examples of chemotherapeutic agents are described in the scientific and patent literature and can be readily determined by those skilled in the art (see, e.g., Bulinski, J. C. et al. (1997) J. Cell Sci. 110:3055-3064; Panda, D. et al. (1997) Proc. Natl. Acad. Sci. USA 94:10560-10564; Muhlradt, P. F. et al. (1997) Cancer Res. 57:3344-3346; Nicolaou, K. C. et al. (1997) Nature 387:268-272; Vasquez, R. J. et al. (1997)Mol. Biol. Cell. 8:973-985; Panda, D. et al. (1996) J. Biol. Chem. 271:29807-29812). Examples of some classes of chemotherapeutic and anti-cancer agents include, but are not limited to, the following: alkylating agents, anti-EGFR antibodies, anti-Her-2 antibodies, antimetabolites, vinca alkaloids, anthracyclines, topoisomerases, taxanes, epothilones, antibiotics, immunomodulators, immune cell antibodies, interferons, interleukins, HSP90 inhibitors, anti-androgens, antiestrogens, anti-hypercalcaemia agents, apoptosis inducers, Aurora kinase inhibitors, Bruton's tyrosine kinase inhibitors, calcineurin inhibitors, CaM kinase II inhibitors, CD45 tyrosine phosphatase inhibitors, CDC25 phosphatase inhibitors, cyclooxygenase inhibitors, cRAF kinase inhibitors, cyclin dependent kinase inhibitors, cysteine protease inhibitors, DNA intercalators, DNA strand breakers, E3 ligase inhibitors, EGF pathway inhibitors, farnesyltransferase inhibitors, Flk-1 kinase inhibitors, glycogen synthase kinase-3 inhibitors, histone deacetylase inhibitors, I-kappa B-alpha kinase inhibitors, imidazotetrazinones, insulin tyrosine kinase inhibitors, c-Jun-N-terminal kinase inhibitors, mitogen-activated protein kinase inhibitors, MDM2 inhibitors, MEK inhibitors, MMP inhibitors, mTor inhibitors, NGFR tyrosine kinase inhibitors, p38 MAP kinase inhibitors, p56 tyrosine kinase inhibitors, PDGF pathway inhibitors, phosphatidylinositol-3-kinase inhibitors, phosphatase inhibitors, protein phosphatase inhibitors, PKC inhibitors, PKC delta kinase inhibitors, polyamine synthesis inhibitors, proteasome inhibitors, PTP1B inhibitors, SRC family tyrosine kinase inhibitors, Syk tyrosine kinase inhibitors, Janus (JAK-2 and/or JAK-3) tyrosine kinase inhibitors, retinoids, RNA polymerase II elongation inhibitors, Serine/Threonine kinase inhibitors, sterol biosynthesis inhibitors, VEGF pathway inhibitors, immunosuppressive agents, CYP3A4 inhibitors, anti-microbial agents, and antiemetics.

In some embodiments, the additional agent is an anti-cancer agent. In certain embodiments, the anti-cancer agent is a MEK inhibitor. As used herein, the term “MEK inhibitor” relates to a compound which (1) targets, decreases or inhibits the kinase activity of MAP kinase, MEK; or (2) disrupts the binding of copper to MEK1 (e.g., blocking the binding site of copper to MEK, inducing/promoting a conformational change of the copper binding site on MEK, etc.). A target of a MEK inhibitor includes, but is not limited to, ERK. An indirect target of a MEK inhibitor includes, but is not limited to, cyclin D1. Examples of suitable MEK inhibitors include, but are not limited to, the following: butanedinitrile; GSK1120212; XL518; selumetinib 6-(4-bromo-2-chloroanilino)-7-fluoro-N-(2-hydroxyethoxy)-3-methylbenzimidazole-5-carboxamide; bis[amino[2-aminophenyl)thio]methylene]-(9Cl); PD184325 (N-(3-chloro-4-fluorophenyl)-7-methoxy-6-(3-morpholin-4-ylpropoxy)quinazol-in-4-amine); PD0325901 (N-[(2R)-2,3-dihydroxypropoxy]-3,4-difluoro-2-[(2-fluoro-4-iodophenyl)amino]-benzamide); PD98059 (2′-amino-3′-methoxyflavone); U0126 (1,4-diamino-2,3-dicyano-1,4-bis(aminophenylthio)butadiene); AZD6244 (6-(4-Bromo-2-chlorophenylamino)-7-fluoro-3-methyl-3H-benzoimidazole-5-carboxylic acid (2-hydroxyethoxy)-amide (described in WO 03/077914, the contents of which are hereby incorporated by reference in its entirety); 2-(2-fluoro-4-iodophenylamino)-N-(2-hydroxyethoxy)-1,5-dimethyl-6-oxo-1,6-dihydropyridine-3-carboxamide; CI-1040 (2-(2-Chloro-4-iodo-phenylamino)-N-cyclopropylmethoxy-3,4-difluoro-benzamide) (CI-1040 is described in PCT Publication No. WO 99/01426, which is incorporated herein by reference in its entirety); N-[(R)-2,3-Dihydroxy-propoxy]-3,4-difluoro-2-(2-fluoro-4-iodo-pheny-lamino)-benzamide (disclosed in PCT Publication No. WO 02/06213, which is incorporated herein by reference in its entirety). Examples of MEK inhibitors which may disrupt the binding of copper to MEK include, but are not limited to, U0126 (see, e.g., Ishizaki, H. et al. (2010) Disease Models & Mechanisms 3:639-651).

When formulating the pharmaceutical compositions described herein, the clinician may utilize preferred dosages as warranted by the condition of the subject being treated. For example, in one embodiment, the subject may be maintained on a copper reducing diet, and a copper chelator may be administered at a dosing schedule described herein, e.g., once every one, two, three, four, five or six weeks.

Also, in general, the one or more copper chelator, and an optional additional chemotherapeutic agent(s) do not have to be administered in the same pharmaceutical composition, and may, because of different physical and chemical characteristics, have to be administered by different routes. For example, the copper chelator may be administered orally, and the additional chemotherapeutic agent(s) may be administered orally or intravenously. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

The actual dosage of the copper chelator and/or any additional chemotherapeutic agent employed may be varied depending upon the requirements of the subject and the severity of the condition being treated. Determination of the proper dosage for a particular situation is within the skill of the art. Generally, treatment is initiated with smaller dosages which are less than the optimum dose of the compound. Thereafter, the dosage is increased by small amounts until the optimum effect under the circumstances is reached.

In some embodiments, when a copper chelator is administered in combination with one or more additional chemotherapeutic agents, the additional chemotherapeutic agent (or agents) is administered at a standard dose.

In accordance with experience and knowledge, the practicing physician can modify each protocol for the administration of a component (copper reducing diet, copper chelator, and chemotherapeutic agent(s), or radiation) of the treatment according to the individual subject's needs, as the treatment proceeds. The attending clinician, in judging whether treatment is effective at the dosage administered, will consider the general well-being of the subject as well as more definite signs such as relief of disease-related symptoms, inhibition of tumor growth, actual shrinkage of the tumor, or inhibition of metastasis. Size of the tumor can be measured by standard methods such as radiological studies, e.g., CAT or MRI scan, and successive measurements can be used to judge whether or not growth of the tumor has been retarded or even reversed. Relief of disease-related symptoms such as pain, and improvement in overall condition can also be used to help judge effectiveness of treatment.

The following examples are offered by way of illustration and not by way of limitation.

### EXAMPLES

### Example 1: Knockdown of Ctr1 Reduces MAPK and Ras Signaling in Flies

To determine the role of copper and the copper transporter, Ctr1, in the Ras signaling pathway, Ctr1 was knocked down in the prothoracic gland of Drosophila. The fruit fly prothoracic gland is a key organ for controlling body size. Mirth, C. K. et al. (2007) Bioessays 29:344-355.

Drosophila melanogaster stocks and crosses. Phantom Gal4, UAS mCD8::GFP/TM6, Tb flies were from Michael O'Connor, University of Minnesota. (see Truman, M. C. et. al. (2005) Curr. Biol. 15:1796-1807). The UAS-Ctr1A^{RNAi} construct was made and transgenic lines were generated as described in Lee, Y. S. et. al. (2003) Methods 30:322-329 and Roberts, D. B. (1998) Drosophia: A Practical Approach. All other stocks were obtained from the Bloomington Stock Center. All crosses were performed at 25° C. All fly work, including pupal measurements, was done at the Duke University Model Systems Unit.

Pupal length Experiments. Wandering 3^{rd}-instar larvae were placed in separate vials according to genotype and sex for pupariation; genotyping was done on the basis of green fluorescent protein (GFP) expression pattern, as well as the dominant marker Tubby. At pupation, individual images were taken using a Leica MZFL III fluorescence stereomicroscope mounted with a Qimaging Retiga Exi digital camera (QImaging, Surrey, Canada) at the same magnification setting. Length measurements were performed by aligning the micrometer ruler image along the length of the pupal case at defined start and end points.

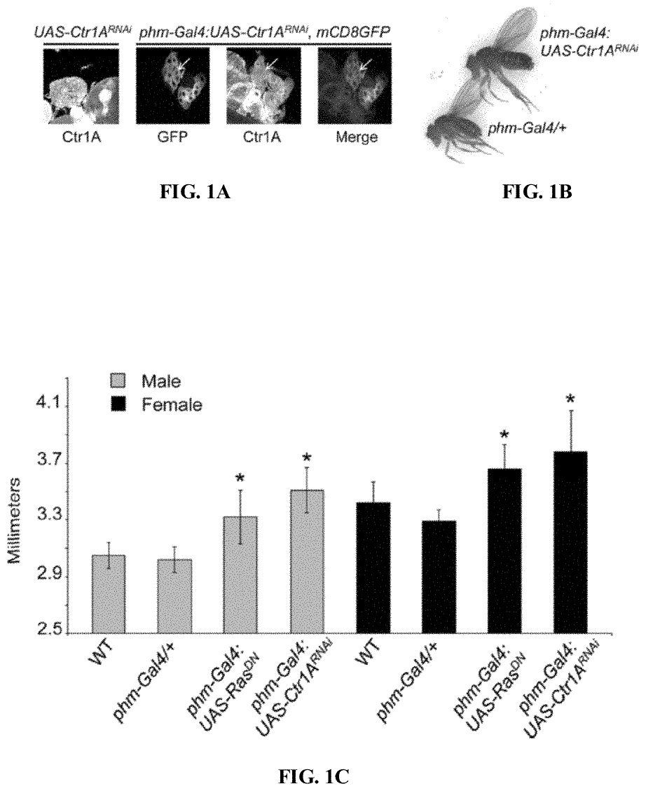

Transgenic flies expressing a yeast Gal4 transcription factor-inducible double-stranded RNA hairpin molecule against Ctr1A (UAS-Ctr1A^{RNAi}) were crossed to flies expressing phantom-Gal4 (phm-Gal4), which drives Gal4 transcription factor expression specifically in the prothoracic gland, resulting in organ-specific reduction of plasma membrane-localized Ctr1A levels. (FIG. 1A). Flies carrying both the UAS-Ctr1A^{RNAi} and phm-Gal4 transgenes are larger than siblings carrying either transgene alone (FIG. 1B) and quantitative measurements of pupae confirmed the increase in size observed in adult flies with a prothoracic-gland-specific Ctr1A knockdown (FIG. 1C). Similar results were obtained with fly stocks in which the UAS-Ctr1A^{RNAi} transgene was integrated on a different chromosome, indicating that this phenotype is not due to a locus-specific integration of the transgene.

Indirect immunofluorescence and scanning electron microscopy (SEM) images. Brains from wandering 3rd-instar Drosophila larvae of the desired genotype were dissected and fixed in 4% paraformaldehyde for 30 minutes. Staining of tissue was performed as described in Turski, M. L. (2007) J. Biol. Chem. 282:24017-24026. Images were taken on a Zeiss LSM 410 confocal microscope at the Duke University Light Microscopy Core Facility (Durham, United States). For SEM images, adult flies of the desired genotype were subjected to a graded ethanol series. Flies were given to the Duke University Shared Materials Instrumentation Facility (Durham, United States) for critical-point drying and sputter coating. SEM images were taken at the Duke University Shared Materials Instrumentation Facility (Durham, United States).

Appropriate Ras protein signaling in the prothoracic gland is critical for body size determination, as constitutively active Ras mutants give rise to small flies while mutations that suppress Ras signaling give rise to abnormally large flies. (FIG. 1C; Caldwell, P. E. (2005) Curr. Biol. 15:13581-13587). Knockdown of the Ctr1A in the prothoracic gland phenocopies the large-fly of prothoracic-gland-specific dominant negative Ras expression, suggesting an interaction between Ctr1A and Ras signaling in the regulation of body size of Drosophila.

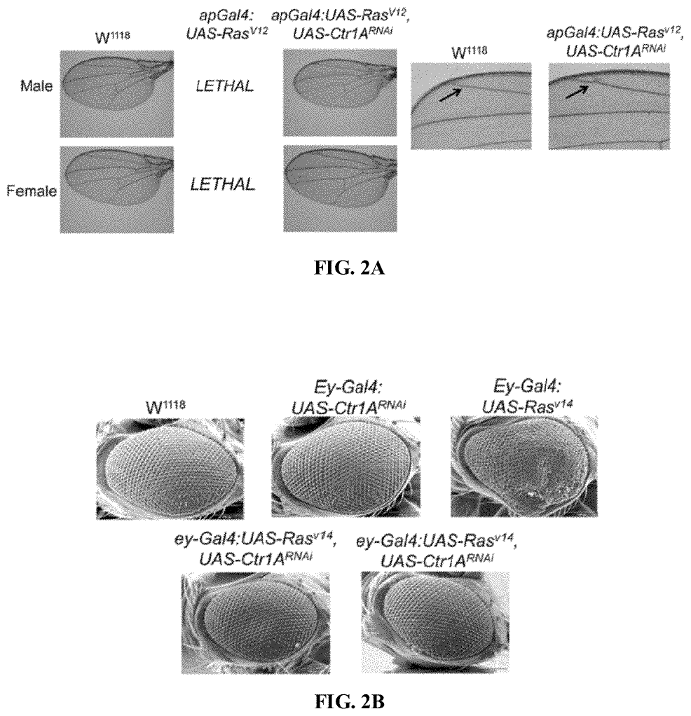

To test the relationship between Ct1A, Ras signaling, and fly body size, Ctr1A knockdown experiments were conducted in flies expressing a constitutively active Ras allele. While expression of constitutively active Ras^{V12} in transgenic flies via the apterous-Gal4 driver (ap-Gal4), which drives expression in the dorsal compartment of the wing, is lethal, coexpression of the UAS-Ras^{V12} and the UAS-Ctr1A^{RNAi} transgenes via ap-Gal4 rescues this lethality and yields viable adult flies with normal wings. In some wings from viable flies, ectopic veins within the posterior compartment of the marginal cell were observed. (FIG. 2A, right panel). This phenotype is also observed in Ellipse mutants possessing hyperactive alleles of the epidermal growth factor receptor that drive increased Ras signaling (see Baker, N. E. (1992) Dev. Biol. 150; 381-396) and indicates a partial suppression of ectopic Ras signaling by Ctr1A knockdown. Moreover, expression of the UAS-Ras^{V14} transgene in the eye using eyeless-Gal4 (ey-Gal4), which yields a rough-eye phenotype characterized by fused ommatidia and disorganized bristles, was suppressed in flies with simultaneous expression of UAS-Ctr1A^{RNAi} and UAS-Ras^{V14}. (FIG. 2B). DsRNA knockdown of Ctr1 in the Drosophila eye rescued the rough-eye phenotype induced by activated Ras (see, e.g., FIG. 2B and Turski, M. L. et al. (2012) supra). Taken together, these data support a genetic interaction between Ctr1A and Ras that occur in multiple Drosophila tissues.

The knockdown of Ctr1A in S2 cells resulted in downregulation of Ras pathway activation to an extent comparable to that achieved by knockdown of canonical pathway members such as the insulin receptor or Ras. Further, reduction of Ctr1A protein levels in S2 cells resulted in decreased Erk phosphorylation.

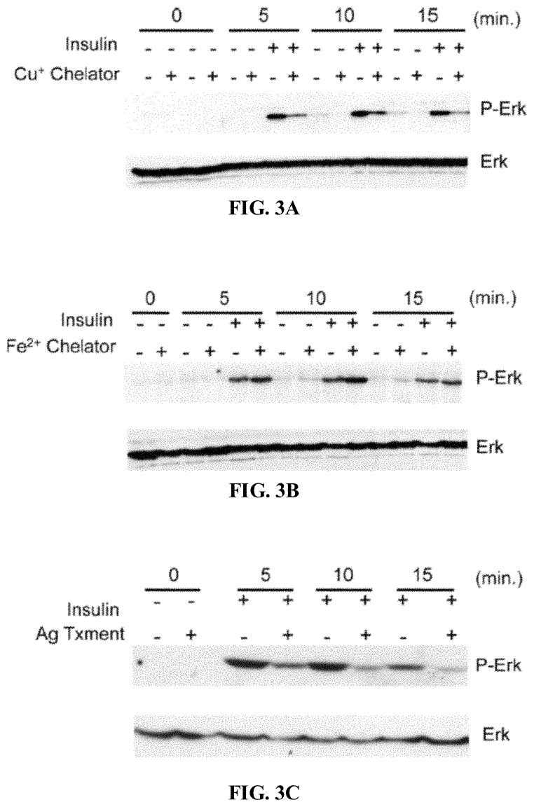

The copper transporter Ctr1 was identified as one of approximately 300 genes that when knocked down in S2 insect cells reduced phosphorylation of Erk (see, e.g., Friedman, A. and Perrimon, N. (2006) Nature 444:230-234). To explore whether both Ctr11 and the associated Cu⁺ transport function are important for Ras signaling to Erk1/2, Cu⁺-specific chelation was used to impose copper deficiency on cultured fly S2 cells, S2 cells used for the no-treatment and insulin-only treatment conditions were left in basal medium (Schneider's medium with 10% fetal bovine serum) during the preincubation. S2 cells used for the other treatment conditions were preincubated for 1 hour with chelator or silver as follows: 10 μM tetrathiomolybdate (TTM) and 250 μM bathocupronine disulfonate (BCS) for copper chelation experiments, 10 μM ferrozine, and 250 μM bathophenanthroline disulfonate (BPS) for iron chelation, and 10 μM silver nitrate. Cells were stimulated with human insulin at a concentration of 25 μg/mL of medium. Cu⁺ chelation reduced the levels of insulin-stimulated Erk1/2 phosphorylation without altering steady-state Erk1/2 levels. (FIG. 3A). This reduction of Erk1/2 phosphorylation by copper chelation was not due to the chelation of all redox-active metals, as the Fe²⁺-specific chelator BPS or ferrozine did not reduce insulin-stimulated Erk1/2 phosphorylation. (FIG. 3B). Moreover, as Ag is isoelectric to Cu⁺ and is a competitive inhibitor of Cu⁺ update transporters, preincubation of S2 cells with Ag clearly diminished the levels of insulin-stimulated Erk1/2 phosphorylation. (FIG. 3C).

### Example 2: Copper is a Co-Factor of MEK Kinases

In the Example presented herein, Mek1 is demonstrated to bind directly to copper and copper is required for Mek1-mediated phosphorylation of Erk1 in vitro. Ctr1^{+/+} and Ctr1^{-/-} mouse embryonic fibroblasts (MEFs) and insulin or fibroblast growth factor (FGF) stimulation experiments. To determine whether copper plays a role in the activation of Erk1/2, which is phosphorylated by Mek1, Ctr1^{+/+} and Ctr1^{-/-} mouse embryonic fibroblasts (MEFs) were evaluated for insulin-stimulated ERK1/2 phosphorylation. Isolation and culture of Ctr1^{+/+} and Ctr1^{-/-} cells were done as described in Lee, J. et al. (2002) J. Biol. Chem. 30:322-329. Insulin or fibroblast growth factor (FGF) stimulation experiments were done with plates measuring 100 by 200 nm, with one plate per time point. Cells were allowed to reach ˜95% confluence and then serum starved for 16 to 48 hours. Recombinant human insulin (Invitrogen, Carlsbad, United States) was added at a final concentration of 200 nM, and recombinant human basic FGF (Invitrogen, Carlsbad, United States) was added at a final concentration of 10 ng/ml, with the exception of the time zero plate. At the appropriate time point, medium was removed, and cells were washed with ice-cold phosphate-buffered saline (PBS), harvested, and lysed using the phosphorylation lysis buffer described above or radio-immunoprecipitation assay (RIPA) buffer consisting of 1% nonylphenoxypolyethoxylethanol (NP-40), 20 mM Tris-HCl (pH 8.0), 137 mM sodium chloride (NaCl), 10% glycerol, 10 mM sodium orthovanadate (Na₃VO₄), 50 mM sodium fluoride (NaF), 50 mM β-glycerophosphate (β-GP), and 1× protease inhibitor cocktail (BD BioSciences, San Jose, United States).

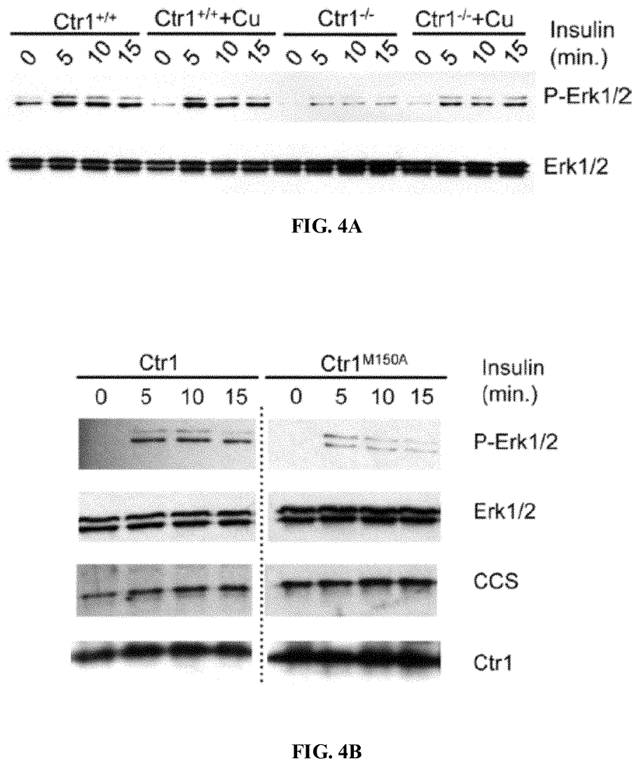

Ctr1^{+/+} MEFs demonstrated a strong insulin-stimulated Erk1/2 phosphorylation within 5 minutes of treatment that was maintained over a 15 minute time course. (FIG. 4A) In contrast, Ctr1^{-/-} MEFs showed only marginal insulin-stimulated Erk1/2 phosphorylation. While Ctr1^{-/-} MEFs exhibit strong reductions in the activity of Cu-dependent enzymes, such as cytochrome oxidase and lysyl oxidase, these activities can be partially rescued by exogenous copper. (Lee, J. (2002) J. Biol. Chem. 277:40253-40259). Preincubation of Ctr1^{-/-} MEFs with 25 μM copper for 1 hour prior to insulin stimulation resulted in increased insulin-stimulated Erk1/2 phosphorylation, though not to the same levels as Ctr1^{+/+} MEFs. (FIG. 4A). No additional stimulation was observed in Ctr1^{+/+} cells when copper was added. These results demonstrate that insulin stimulation of Erk1/2 phosphorylation in mammalian cells is heavily dependent on Ctr1 and that, in the absence of Ctr1, this defect can be partially ameliorated by exogenous copper.

### Generation of Ctr1^{-/-}: CMV-Ctr1 and Ctr1^{+/+}: CMV-Ctr1^{M150A} Stable Cell Lines

Previous studies demonstrated that two methionine residues located in the second transmembrane domain of Ctr1 in a Met-X3-Met motif are important for Ctr1-mediated Cu⁺ transport but not for oligomerization or localization to the plasma membrane. (Puig, S. et al. (2002) J. Biol. Chem. 277:26021-26030). To determine if the integrity of this motif is important for insulin-stimulated Erk1/2 phosphorylation, Ctr1^{-/-} l MEFs were stably transfected with plasmids expressing either wild-type human Ctr1 or Ctr1 in which the first methionine in this motif, M150, had been altered to alanine and evaluated for insulin-stimulated Erk1/2 phosphorylation (FIG. 4B). The Ctr1 and Ctr1^{M150A} coding sequences were PCR amplified using plasmid templates described in Puig, S. et al. (2002) J. Biol. Chem. 277:26021-26030, and cloned into the pcDNA3.1(+) Zeocin vector (Invitrogen, Carlsbad, United States). MEFs genetically null for Ctr1 (see Lee, J. et al. (2002) Proc. Natl. Acad. Sci. U.S.A. 277:40253-40259) were electroporated with these constructs using the Amaxa Nucleofector kit in accordance with the manufacturer's recommendations. Stable cell lines were generated according to standard protocols (Animal Tissue Culture Book), and Zeocin resistance was used as the selective marker.

While the Ctr1^{-/-} cells rescued with wild-type Ctr1 showed robust insulin-induced Erk1/2 phosphorylation, this was strongly reduced in MEFs stably expressing the Cu transport-defective Ctr1^{M150A} protein. Although both Ctr1 wild-type and Ctr1^{M150A} MEFs expressed approximately equivalent amounts of Ctr1, the Ctr1^{M150A} cells remained more Cu deficient, as indicated by the increased steady state levels of CCS, which is subject to ubiquitin-mediated proteolysis in the presence of elevated Cu levels and stabilized during Cu deficiency. (See Caruano-Yzermans, A. L. (2006) J. Biol. Chem. 281:13581-13587). Taken together with the findings on Cu chelation, Ag competition, and exogenous Cu rescue of Ctr1^{-/-} MEFs, these results strongly suggest that Cu and the Cu⁺-transporting activity of Ctr1 are important for normal activation of Erk1/2 phosphorylation in flies and mice.

Genetic and biochemical experiments demonstrated the involvement of Ctr1A in flies and Ctr1 in mammals in the Ras-to-Erk signaling pathway. To test whether Ras represents the key intersection point for Ctr1 and copper, and thus whether Ctr1 and Cu alter the activity of multiple signaling pathways downstream of Ras, the Ras/PI3K/Akt kinase signaling pathway. Protein was evaluated quantified using the Bio-Rad DC protein assay and run on precast Criterion Tris-HCl polyacrylamide gradient gels (Bio-Rad, Hercules, United States) or 10% SDS-PAGE. The primary antibodies used are as follows: mouse anti-BRaf, mouse anti-Mek1, rabbit anti-Mek2, rabbit anti-Erk2, mouse anti-Mek1/2, rabbit anti-p44/42MAPK(Erk1/2), rabbit anti-Akt, rabbit anti-phospho-Mek1/2 (Ser217/221), mouse anti-phospho-p44/42 MAPK (Erk1/2) (Thr202/Tyr204), rabbit anti-phosphop44/42 MAPK (Erk1/2) (Thr202/Tyr204), and rabbit anti-phospho-Akt (Thr308) (Cell Signaling Technology, Danvers, United States) used at a 1:1,000 dilution; goat anti-phospho-BRaf (Thr598/Ser601) (1:500 dilution) and rabbit anti-CCS (anti-copper chaperone for superoxide dismutase 1; FL-274) (Santa Cruz Biotechnology, Santa Cruz, United States) used at 1:200 dilution; rat anti-myelin basic protein (anti-MBP) and mouse anti-phospho-MBP (Millipore, Billerica, United States) used at 1:500 dilution; rabbit anti-kinase suppressor of Ras (anti-KSR) (Abcam, Cambridge, United States) used at 1:500 dilution; mouse anti-β-actin (Sigma-Aldrich, St. Louis, United States) used at 1:25,000 dilution); the rabbit anti-human Ctr1 antibody, described in Nose, Y. et al. (2006) Cell Metab. 4:235-244, was used at 1:1,000. Secondary antibodies were donkey anti-rabbit and anti-mouse antibodies conjugated with (GE Healthcare Life Sciences) used at 1:5,000 dilution or goat anti-mouse IgG (Invitrogen, Carlsbad, United States) used at 1:10,000 dilution, goat anti-mouse IgG light chain specific (Jackson ImmunoResearch Laboratories, West Grove, United States) used at 1:5,000 dilution, goat anti-rabbit (Invitrogen, Carlsbad, United States) used at 1:10,000 dilution, mouse anti-rabbit IgG light chain specific (Jackson ImmunoResearch Laboratories, West Grove, United States) used at 1:5,000 dilution, goat anti-rat IgG (Zymed) used at 1:10,000 dilution, and rabbit anti-goat IgG (Invitrogen, Carlsbad, United States) used at 1:5,000 dilution conjugated with horseradish peroxidase. Metal chelate affinity purification experiments were performed as described in Mufti, A. R. et al. (2006) Mol. Cell. 21:775-785.

No significant changes in phosphorylation at Thr308 of Akt1, which is the key residue phosphorylated by PDK1 in response to PI3K pathway activation (Alessi, D. R. et al. (1996) EMBO J. 15:6541-6551) in either the Ctr1^{+/+} or the Ctr1^{-/-} cell line. These results suggest that the Ctr1 and Cu-responsive components of Ras signaling lie downstream of Ras and do not impact the Ras/PI3K/AKT signaling network. (See Turski, M. L. (2012), supra).

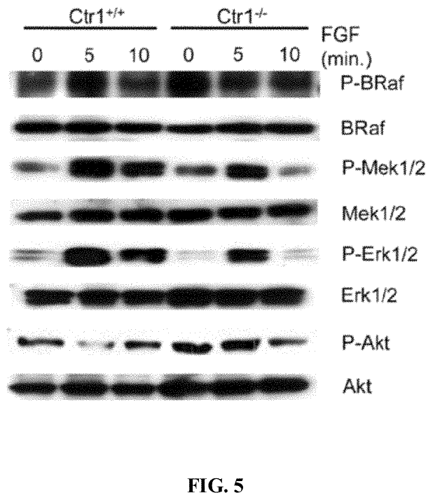

To determine whether Copper influences the Ras/Raf/Mek/Erk signaling pathway, the steady-state levels and phosphorylation status of components of this pathway downstream of FGF-stimulated Ras activation were evaluated in Ctr1^{+/+} and Ctr1^{-/-} cells by immunoblotting (FIG. 5). Activation of the main Raf kinase in MEFs, B-Raf (Dougherty, M. K. et al. (2005) Mol. Cell 17:215-224), occurred to a similar extent in both Ctr1^{+/+} and Ctr1^{-/-} cells as assessed by evaluating phosphorylation of Thr598 and Ser601 in B-Raf, two key residues that become phosphorylated upon Ras activation (Zhang, B. H. (2000) EMBO J. 19:5429-34035). Increased phosphorylation of B-Raf on Thr598 and Ser601 occurred in unstimulated Ctr1^{-/-} l MEFs. The increase in phosphorylation in the knockout versus wild-type MEFs is due to the absence of active Erk1/2-mediated negative feedback on the MAPK signaling pathway that disrupts Raf-1/B-Raf dimerization. (See Rushworth, L. K. et al. (2006) Mol. Cell. Biol. 26:2262-2272). Given the similar levels of Akt phosphorylation in Ctr1 wild-type versus Ctr1 knockout cells, Ras activity is not affected by loss of Ctr1 or reductions in intracellular Cu levels. Active Ras binds to and activates the Raf kinases that phosphorylate and activate the serine threonine MAPK kinases Mek1 and Mek2. Phosphorylation of Mek1 and Mek2 is observed in both Ctr1^{+/+} and Ctr1^{-/-} cells, demonstrating that Raf activity is not affected by loss of Ctr1 or reductions of intracellular Cu levels. Activated Mek1/2 phosphorylate Erk1 and Erk2, and signal transduction events downstream of Erk ultimately result in the dephosphorylation and inactivation of Mek1/2. (Kolch W. (2000) Biochem J. 351(Pt. 2):289-305; Shaul, Y. D. (2007) Biochem. Biophys. Acta. 1773:121-1226). As observed previously upon insulin stimulation, FGF-stimulated phosphorylation of Erk1/2 was diminished in Ctr1^{-/-} cells compared to that in cells expressing Ctr1, consistent with a defect in Erk1/2 activation (FIG. 5). Similar effects on Ras/MAPK pathway activation were also obtained when insulin was used as the stimulus. The results of this study demonstrate that loss of the Ctr1 Cu⁺ transporter or reductions in Cu accumulation result in a diminution of Erk1/2 phosphorylation without altering the upstream signatures of Raf activation. Thus, Cu plays a role in the Ras/MAPK signaling pathway at the juncture where Mek1/2 phosphorylates Erk1/2.

To determine whether MEK1 itself may be a Cu-binding protein, extracts from wild-type MEFs were incubated with beads conjugated with metal-binding tripeptide GSH that was either uncharged or charged with Cu. Mek1 protein was expressed in and purified from Escherichia coli and applied to pentadentate beads for Mek1 partitioning and immunoblotting experiments. Metal pulldown experiments were conducted as described in Mufti, A. R. (2006) Mol. Cell 21:775-785. Metal pulldown experiments were conducted by loading 100 μg of protein into the input lane and 500 μg of protein lysate was incubated with the glutathione (GSH)-copper beads. After one hour incubation, the lysate was removed, the beads were washed several times, Laemmli buffer was added to the beads, the samples were boiled, and the entire sample volume was loaded onto the gel.

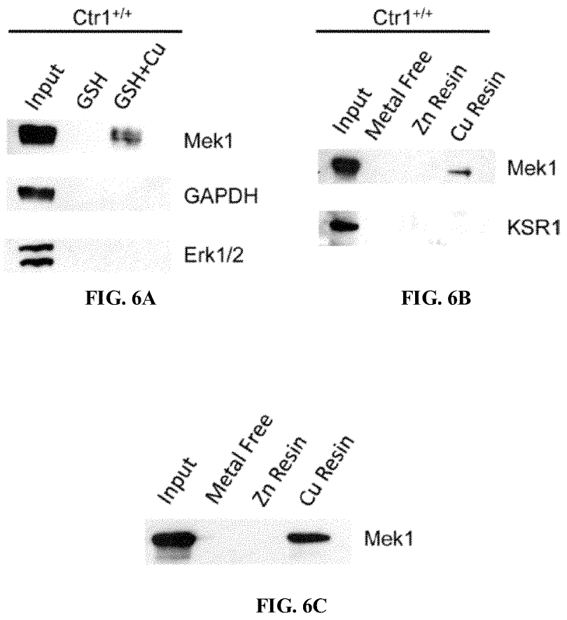

Results demonstrated that GSH beads alone were unable to purify Mek1, Erk1/2, or glyceraldehyde 3-phosphate dehydrogenase (GAPDH) from the lysate. (See FIG. 6A, Turski, M. L. et al. (2012), supra). However, when the GSH beads were preloaded with Cu, Mek1, but not Erk1/2 or GAPDH, was enriched from total cell extracts. To further test the specificity of this metal-binding resin and the metal specificity of Mek1 binding, the broad-spectrum metal-binding ligand pentadentate was used in affinity purification with MEF protein extracts. While metal-free or Zn-loaded pentadentate beads were unable to purify Mek1 protein, Mek1 was partitioned from the extract with Cu²⁺-loaded pentadentate. (FIG. 6B). Moreover, the Mek1/2 scaffold protein KSRI (Kolch, W. (2005) Nat. Rev. Mol. Cell Biol. 6:827-837) was not bound to Cu-pentadentate. Additionally, recombinant Mek1 could be absorbed onto copper-loaded pentadentate beads but not metal free or Zn-loaded beads. (FIG. 6C). These results demonstrate that Mek1 directly interacts with copper, and has the ability to discriminate between copper and zinc.

To investigate the nature of copper binding to Mek1, purified recombinant Mek1 was used for in vitro copper binding stoichiometry and binding affinity experiments. In vitro copper-binding experiments were carried out using the rat Mek1 coding sequence, which was PCR amplified using the pCMV-HAMek1 construct and cloned into the pGex6P-1 vector (GE Healthcare Life Sciences, Waukesha, United States). The resulting plasmid, pGex69-1Mek1, was transformed into BL21-CodonPlus (DE3)-RIPL cells from Stratagene. Recombinant, glutathione S-transferase (GST)-tagged Mek1 was purified by affinity chromatography using GSH agarose beads, followed by on-column Pre Scission protease cleavage of the GST tag. Further purification was achieved using MonoQ anion-exchange chromatography that served to remove the majority of the contaminating proteins, including cleaved GST tags that were not retained on the GSH column. SDS-PAGE of the resulting Mek1 revealed a predominant single band at ˜44 kDa. Protein concentrations were determined by quantitative amino acid analysis with a Beckman 6300 analyzer (Beckman Coulter, Brea, United States) after hydrolysis in 5.7 N HCl at 110° C. in vacuo. Equilibrium dialysis experiments were conducted as described in Horng, Y. C. et al. (2006) J. Biol. Chem. 280:34113-34122. Recombinant Mek1 (2.5 to 10 μM in 20 mM Tris [pH 7.2]) was dialyzed, using a dialysis tube with 10-kDa molecular mass cutoff, against CuCl₂ concentrations ranging from 0.25 to 15 μM in 20 mM Tris (pH 7.2)-100 mM NaCl overnight at 4° C. with slow stirring. Copper levels associated with Mek1 and the dialysate were quantified by inductively coupled plasma mass spectroscopy after digestion with 50% HNO₃ at 65° C. In specific experiments, Cu²⁺ was introduced as a Cu²⁺-histidine complex (His complex) to preclude Cu²⁺ hydrolysis and precipitation.

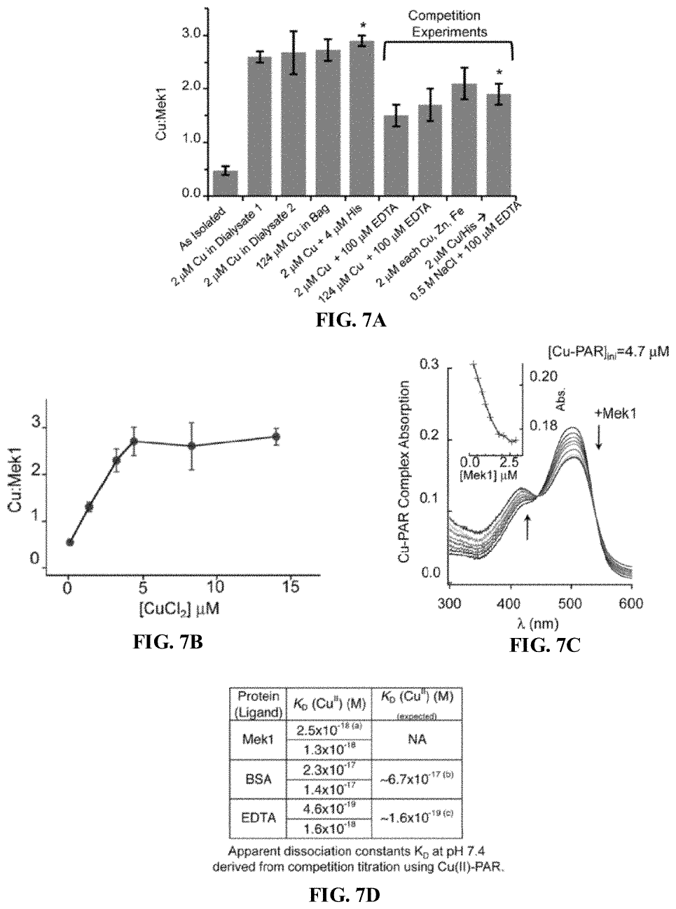

As shown in FIG. 7A, dialysis against 2 μM Cu²⁺ increased the bound copper contend to ˜2.5 molar equivalents from the as-isolated ratio of ˜0.5. To prevent metal hydrolysis by water, Cu was also introduced as a Cu-His complex in the dialysate; this did not alter the bound Cu content. However, the inclusion of 0.1 mM EDTA reduced the Cu content to ˜1.5 molar equivalents. Dialysis of Mek1 against 2 μM Cu²⁺-4 μM His complex, followed by a subsequent dialysis step in 0.1 mM EDTA, reduced the copper content to approximately 2 molar equivalents. Inclusion of Zn(II) and Fe(II) as pared to 2.6 when only Cu²⁺ was present. Taken together, these dialysis experiments demonstrate specific high-affinity Cu²⁺-binding sites in Mek1 and a site with low-affinity interaction. In a separate series of equilibrium dialysis experiments in which the Cu²⁺ content was varied from 0.11 to 0.15 μM, the maximal copper content associated with Mek1 plateaued near ˜2.5 molar equivalent (FIG. 7B). Both dialysis experiments demonstrate more than one copper binding site on Mek1. Under these same conditions, Cu²⁺ binding is observed with albumin, a known Cu²⁺-binding protein, but not lysozyme or thyroglobulin.

To obtain a precise binding affinity, a series of ligand competition studies using PAR were conducted. PAR is a chromogenic chelator forming colored complexes with metal ions. Cu²⁺ binding affinity for Mek1 was estimated using competition experiments similar to those described in Zimmermann, M. et. al. (2009) Biochemistry 48:11640-11654, with the divalent metal ligand PAR [4-(2-pyridylazo)resorcinol]. The quantitative release of the 1:1 Cu²⁺-/PAR complex on titration of apo-Mek1 was monitored spectrophotometrically at 500 nm (DU 600 spectrophotometer, Beckman Coulter, Brea, United States) in 20 mM Tris (pH 7.2)-100 mM NaCl. The binding affinity of Cu²⁺ for PAR was calibrated using spectroscopically silent ligand, EDTA, with a known affinity for Cu²⁺ of 1.6×10⁻¹⁹.

The affinity of Cu²⁺-PAR complex (formation constant [β]) is 3.2×10¹⁷, and the equilibrium concentration of the complex is measurable at 500 nm (extinction coefficient [ϵ], 35,500 M⁻¹cm⁻¹) with an isobestic point at 445 nm. Bidentate PAR forms a 1:1 complex with Cu²⁺. Titration of apo-Mek1 with the Cu-PAR complex revealed a concentration-dependent attenuation of the Cu²⁺-PAR concentration, consistent with equilibrium of Cu²⁺ from PAR to Mek1 (FIG. 7C). The initial rapid decrease at 500 nm on increasing apo-Mek1 addition is indicative of a strong affinity of Mek1 for Cu²⁺. (FIG. 7C, inset). Titration of apo-Mek1 with the Cu-PAR complex revealed a concentration-dependent attenuation of the Cu²⁺-PAR concentration, consistent with equilibration of Cu²⁺ from PAR to Mek1. The initial rapid decrease at 500 nm on increasing apo-Mek1 addition is indicative of a strong affinity of Mek1 for Cu²⁺. This decrease at 500 nm was observed until the Mek1/Cu ratio reached ˜0.5, which showed that effective competition between the PAR ligand and Mek1 was induced.

Control titrations under the same pH and ionic strength buffer conditions were performed with EDTA and bovine serum albumin (BSA) to validate the Cu²⁺-PAR titration study. Both EDTA and BSA are spectrally silent, with known dissociation constants. EDTA also served to calibrate Cu²⁺-PAR affinity relative to the reaction condition used for the experiments. Calculations of the Cu²⁺-binding affinities of EDTA and BSA confirmed literature values for both ligands. (FIG. 7D). The Mek1 dissociation constant was ˜10⁻¹⁸ M, demonstrating that Cu²⁺ is associated with Mek1. As the Cu²⁺ binding stoichiometry is ˜2.5 molar equivalent, it is unknown whether the Cu²⁺ binding sites are equivalent. However, these results demonstrate that Mek1 binds directly to copper. (FIGS. 6A-6C, FIGS. 7A-7D, and FIG. 8, Turski, M. L. et al. (2012), supra).

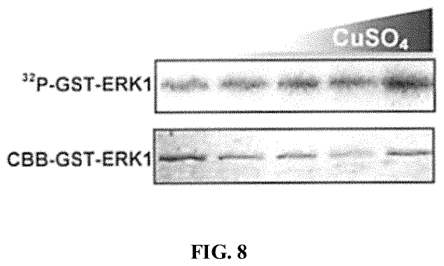

Mek1 requires copper for kinase activity in vitro (FIG. 8 and Turski, M. L. et al. (2012), supra). To determine the role copper plays in the stimulation of Mek1-dependent phosphorylation of Erk1/2, a series of in vitro experiments were carried out. For these experiments, purified human Mek1 protein was incubated with a kinase-dead isoform of hErk2 so that assessment of Erk2 phosphorylation mediated by Mek1 could be made in the absence of Erk2 autophosphorylation. Seger, R. et. al. (1991) Proc. Natl. Acad. Sci. U.S.A. 88:6142-6146.

Human Erk2 and human Mek1 were obtained from Addgene and cloned into pGEX4T3 and pGEX6P1 from GE Life Sciences, respectively. Recombinant GST-human Erk2 (hErk2) and GST-human Mek1 (hMek1) were purified from BL21(DE3) bacteria as previously described. (see Heise, C. J. (2006) Methods 40:209-212; Levin-Salomon, V. et al. (2008) J. Biol. Chem. 283:34500-34510). Specifically, 500 mL of LB was inoculated with BL21(DE3) bacteria transformed with pGEX4T3-hErk2 or pGEX6P1-hMek1 and allowed to grow to an optical density of 0.6 at 600 nm (OD₆₀₀). GST-hErk2 was induced by IPTG (0.4 mM) at 37° C. for 4 hours, while GST-hMek1 was induced by IPTG (1 mM) for 14 hour at 25° C. before collection by ultracentrifugation. GST-hErk1 and GST-hMek1 pellets were resuspended in 50 mL of 1×PBS-1% Triton X-100 plus a protease inhibitor tablet and sonicated for bacterial lysis. The soluble fraction was obtained via ultracentrifugation and incubated with 1 mL of a 50% slurry of GSH-Sepharose 4B overnight at 4° C. with elution buffer (100 mM Tris-HC1 [pH 8.0], 120 mM NaCl) containing 15 mM GSH. Eluted GST proteins were dialyzed in tubing with a 12 to 14,000 molecular weight cutoff overnight at 4° C. in 2 liters of elution buffer and subsequently concentrated using 10K Amicon Ultra Centrifugal filter units (Amicon, Billerica, United States). The concentration was determined using the Bio-Rad DC protein assay (Bio-Rad, Hercules, United States).

Modified version of Mek1 and Erk2 in vitro kinase assay were performed as described previously. (See Kubota, Y. et al. (2011) Nat. Cell Biol. 13:282-291; Levin-Salomon, V. et al. (2008) J. Biol. Chem. 283:34500-34510). Briefly, for Mek1 kinase assays, 0.6 μg of GST-hErk2 and 1.4 μg of GST-Mek1 were incubated in 1804 of kinase buffer (25 mM Tris-HCl [pH 7.5], 20 mM MgCl₂, 2 mM dithiothreitol [DTT], 25 mM β-GP, 0.5 mM Na₃VO₄, 120 μM ATP) in the presence or absence of increasing amounts of CuSO4, 50 μM TTM in the presence of CuSO₄, or 1 μM Mek inhibitor 1 in the presence of CuSO₄ at 22° C. for 30 minutes. Reactions were quenched with 5× Laemmli buffer, and a third of the reaction mixture was analyzed by SDS-PAGE via subsequent Western blotting with phosphospecific antibodies. Briefly, for the Erk2 kinase assays, 2.0 μg of GST-Erk2 and 1.0 μg of MBP were incubated in 180 μL of kinase buffer (25 mM HEPES [pH 8.0], 20 mM MgCl₂, 1 mM DTT, 20 mM β-GP, 0.1 mM Na₃VO₄, 100 mM ATP) at 30° C. for 30 minutes. Reactions were quenched with 5× Laemmli buffer, and a third of the reaction mixture was analyzed by SDS-PAGE via Western blotting with phosphospecific antibodies.

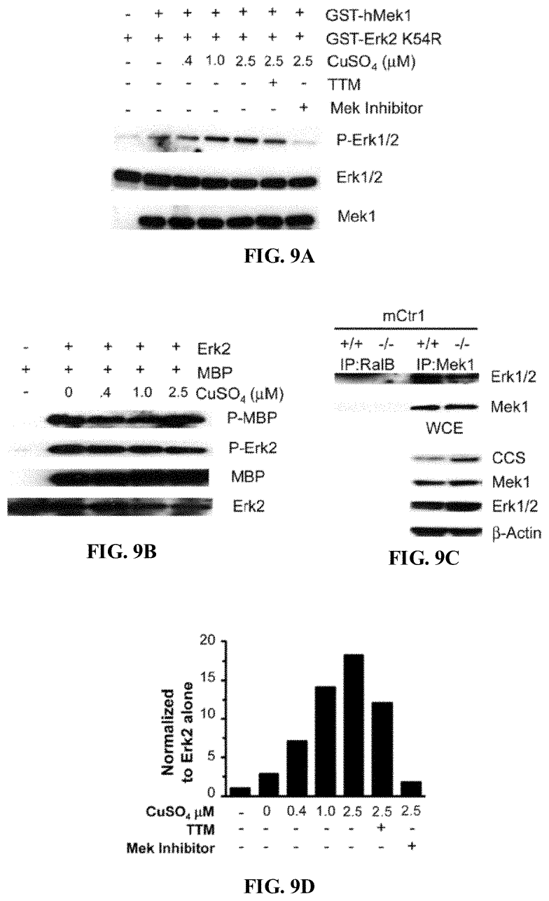

The results in FIG. 9A are representative results of three in vitro kinase activity assays that yielded similar trends for Mek1 activity. When recombinant Mek1 was incubated with kinase-dead Erk2, an ˜2-fold increase in Erk2 phosphorylation was observed that may have been due to residual copper that copurified with recombinant Mek1 protein compared to kinase-dead Erk2 alone which in and of itself still retains some autophosphorylation ability. However, Mek1 kinase activity was greatly enhanced by copper addition in a dose-dependent manner, with Metk1 activity ˜20 times greater in the presence of 2.5 μM CuSO₄. Furthermore, Mek1 activity in the presence of 2.5 μM CuSO₄ was blunted by the addition of TTM, a Cu-chelating agent. Similar in vitro kinase assays were performed with recombinant wild-type Erk2 protein, and no effect of Cu addition on Erk2 phosphorylation of MBP, a commonly used substrate for Erk kinase assay, was observed. (FIG. 9B).

Immunoprecipitation. To determine whether copper triggers the Mek1 phosphorylation of Erk by enhancing the association of those two proteins, coimmunoprecitpitation experiments were used to determine the interaction between endogenous Mek1 and Erk1/2 under Cu-replete (Ctr1^{-/-} MEFs) or Cu-deficient (Ctr1^{+/+} MEFs) conditions. Ctr1^{+/+} and Ctr1^{-/-} lysates were solubilized with the RIPA buffer described above, and the lysates (250 μg) were incubated with anti-Mek1 antibody (1:50) overnight and then with protein G-Sepharose 4B for 2 hours. Beads were washed 3 times in RIPA buffer. Immunoprecipitates were resolved by SDS-PAGE and analyzed by Western blotting with anti-Mek1 and anti-Erk1/2 antibodies. Equal loading was analyzed with whole-cell extract by Western blotting with anti-Mek1, anti-Erk1/2, anti-CCS, and β-actin antibodies. While a fraction of Mek1 and Erk1/2 can be coimmunoprecipitated in Ctr1^{+/+} MEFs, this interaction was significantly reduced in Ctr1^{-/-} MEFs (FIG. 9C). Mek1 kinase activity and association with Erk were shown to be stimulated by Cu (FIG. 9D).

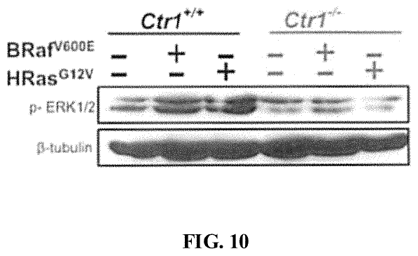

Based on the aforementioned data, it was found that activation of Erk1/2 by oncogenic Ras^{G12V} or BRaf^{V600E} was greatly reduced in mouse embryonic fibroblasts (MEFs) homozygous null for the Ctr1 gene (FIG. 10). Epistatic experiments revealed that the defect lied at the level of Mek1/2. These and other experiments indicate that copper is a co-factor for Mek1/2 activity (see, e.g., Turski, M. L. et al. (2012), supra).

### Example 3. Physiological Role for Ctr1 in Erk Activation in Mice

To test for a potential physiological requirement for Ctr1 in Mek1 function in animals, mice were generated with cardiac-tissue-specific ablation of Ctr1 expression (Ctr1^{hrt/hrt} mice)) as described in Kim, B. E. et al. (2010) Cell Metab. 11:353-363. Mice possessing the Ctr1 gene flanked by loxP elements (Ctr1^{flox/flox}) were described in Nose, Y. et al. (2006) Cell. Metab. 4:235-244. Cardiac tissues from age-matched mice (10 days old) were dissected after perfusion with PBS (pH 7.4) and homogenized in cell lysis buffer (62.5 mM Tris [pH 6.8], 2% sodium dodecyl sulfate [SDS], 1 mM EDTA) containing protease inhibitor cocktail (Roche, Basle, Switzerland) and phosphatase inhibitor cocktail (Thermo Scientific, Waltham, United States). Anti-CCS antibody (Santa Cruz Biotechnology, Santa Cruz, United States) was used at a 1:2,000 dilution. Antitubulin antibody (Sigma-Aldrich, St. Louis, United States) was used at a 1:5,000 dilution.

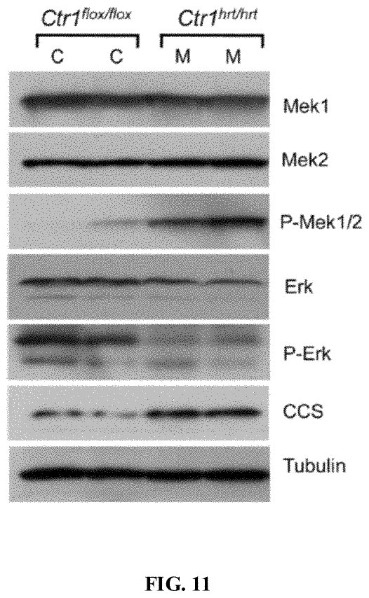

Protein extracts from two control (C) and two Ctr1^{hrt/hrt} mutant (M) littermates were evaluated for Erk1/2 phosphorylation by immunoblotting. As shown in FIG. 11, hearts from the two Ctr1^{hrt/hrt} mice were Cu deficient, as evidenced by the increased steady-state levels of CCS compared to those of wild-type control littermates. Moreover cardiac tissue protein extracts from Ctr1^{hrt/hrt} mice showed a clear reduction in Erk1/2 phosphorylation and a concomitant increase in phospho-Mek1/2 levels compared to those of wild-type littermates. The results from tissue-specific ablation of Ctr1 in mice parallel those observed comparing cultured Ctr1^{+/+} and Ctr1^{-/-} MEFs and demonstrate a clear physiological role for Ctr1 in Mek mediated Erk phosphorylation in mammalian tissues.

### Example 4: BRaf^{V600E} Tumorigenesis Depends Upon Copper

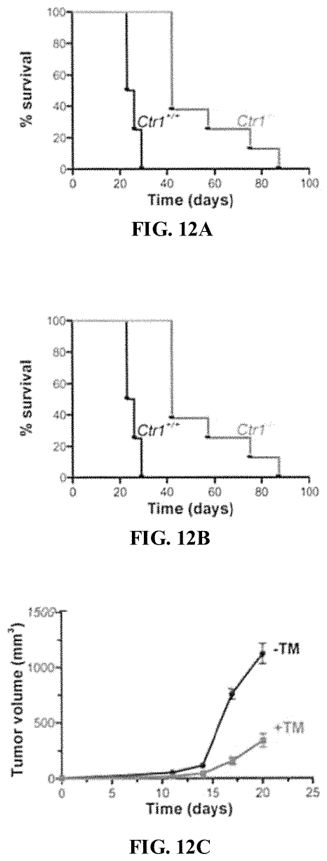

Given the requirement of copper for Mek1/2 activity, BRaf^{V600E}-transformed Ctr1^{+/+} and Ctr1^{-/-} MEFs were injected into mice, revealing that the loss of Ctr1 tripled the time mice took to reach survival endpoints (FIG. 12A). shRNA knockdown of Ctr1 similarly reduced tumor growth of BRaf^{V600E} mutation-positive human melanoma cancer cell lines. Interestingly, knockdown of Ctr1 had no effect on wither NRAS/BRAF mutation-negative human melanoma cell lines or cells transformed with other oncogenes (not shown), indicating that the requirement for copper is specific for BRaf^{V600E}-dependent tumorigenesis. Finally, it was found that knockdown of Mek1 in BRaf^{V600E}-transformed cells reduced their tumorigenic growth, and most importantly, this phenotype could not be rescued by Mek1 copper-binding mutants (FIG. 12B). The data suggests that copper is critical for Mek1/2 to promote oncogenic BRaf-dependent tumor growth.

### Example 5: Wilson's Disease

Copper is provided primarily through diet. This brings up the very exciting possibility that simple dietary changes, coupled with pharmacologic approaches to reduce copper levels and hence Mek1/2 kinase activity, could be used to enhance the anti-tumor activity of the BRaf^{V600E} kinase inhibitors for the treatment of metastatic melanoma. Similar copper-reducing strategies may even hold promise as a way to preemptively reduce the incidence of melanoma in high-risk populations. In this regards, there are well-established approaches to regulate the level of copper in humans. Specifically, Wilson's Disease is characterized by a mutation in the copper-transporting gene ATP7B that results in elevated levels of copper in the body (see, e.g., Das, S. K. and Ray, K. (2006) Nat. Clin. Pract. Neurol. 2:482-493). This disease is treated by first lowering copper levels with copper chelators D-penicillamine, trientine or investigative drugs such as ammonium tetrathiomolybdate (TM). Copper levels are then maintained by a copper-restricted diet (e.g., avoidance of copper-rich foods such as shellfish, nuts, chocolate, liver and cooking in copperware) and either zinc acetate, to block copper absorption, or low dose copper chelators (see, e.g., Das, S. K. and Ray, K. (2006), supra). To evaluate if reducing dietary copper could negatively impact melanoma, mice injected with BRaf^{V600E}-transformed MEFs were either untreated as a control or treated with oral TM to chelate dietary copper. At the termination of the experiment tumors in mice treated with TM were nearly five times smaller than the control tumors (FIG. 12C), thereby suggesting that reducing dietary copper inhibits BRaf^{V600E}driven tumorigenesis.

Any patents or publications mentioned in this specification are indicative of the levels of those skilled in the art to which the invention pertains. These patents and publications are herein incorporated by reference to the same extent as if each individual publication was specifically and individually indicated to be incorporated by reference.

One skilled in the art will readily appreciate that the present invention is well adapted to carry out the objects and obtain the ends and advantages mentioned, as well as those inherent therein. The present examples along with the methods described herein are presently representative of preferred embodiments, are exemplary, and are not intended as limitations on the scope of the invention. Changes therein and other uses will occur to those skilled in the art which are encompassed within the spirit of the invention as defined by the scope of the claims.

1-25. (canceled)

26. A method of inhibiting oncogenic BRaf-dependent tumor growth in a subject in need thereof, said method comprising administering to the subject a pharmaceutical composition that is capable of inhibiting the copper-dependent kinase activity of at least one of MEK1 and MEK2.

27. The method of claim 26, wherein said oncogenic BRaf-dependent tumor comprises BRaf^{V600E}.

28. The method of claim 26, wherein said pharmaceutical composition reduces copper levels in cells of said oncogenic BRaf-dependent tumor or prevents uptake of copper by said subject.

29. The method of claim 28, wherein said pharmaceutical composition is a copper chelator.

30. The method of claim 29, wherein said copper chelator is selected from the group consisting of penicillamine, bathocuprione sulfonate, sodium diethyldithiocarbamate, trientine hydrocholoride, dimercaprol, ammonium tetrathiomolybdate (TM), and zinc acetate, or a combination thereof.

31. The method of claim 28, wherein said pharmaceutical composition reduces the expression of CTR1 copper transporter or knockdowns the gene encoding the CTR1 copper transporter.

32. The method of claim 26, said method further comprising co-administering a copper reducing diet to the subject.

33. The method of claim 32, wherein said copper reducing diet is a diet that is avoidance of copper-rich food and not cooked in copperware.

34. The method of claim 32, said method further comprising administering in combination with a therapeutic treatment modality.

35. The method of claim 34, wherein said therapeutic treatment modality is a surgery, a radiation therapy, a cryosurgery, a thermotherapy, or a combination thereof.

36. The method of claim 32, said method further comprising administering in combination with at least one additional therapeutic agent.

37. The method of claim 36, wherein said at least one additional therapeutic agent is a chemotherapeutic agent, an anti-cancer agent, or a combination thereof.

38. The method of claim 37, wherein said at least one additional therapeutic agent is selected from the group consisting of an alkylating agent, an anti-EGFR antibody, an anti-Her-2 antibody, an antimetabolite, a vinca alkaloid, an anthracycline, a topoisomerase, a taxane, an epothilone, an antibiotic, an immunomodulator, an immune cell antibody, an interferon, an interleukin, a HSP90 inhibitor, an anti-androgen, an antiestrogen, an anti-hypercalcaemia agent, an apoptosis inducer, an Aurora kinase inhibitor, a Bruton's tyrosine kinase inhibitor, a calcineurin inhibitor, a CaM kinase II inhibitor, a CD45 tyrosine phosphatase inhibitor, a CDC25 phosphatase inhibitor, a cyclooxygenase inhibitor, a cRAF kinase inhibitor, a cyclin dependent kinase inhibitor, a cysteine protease inhibitor, a DNA intercalator, a DNA strand breaker, an E3 ligase inhibitor, an EGF pathway inhibitor, a farnesyltransferase inhibitor, a Flk-1 kinase inhibitor, a glycogen synthase kinase-3 inhibitor, a histone deacetylase inhibitor, an I-kappa B-alpha kinase inhibitor, an imidazotetrazinone, an insulin tyrosine kinase inhibitor, a c-Jun-N-terminal kinase inhibitor, a mitogen-activated protein kinase inhibitor, a MDM2 inhibitor, a MEK inhibitor, a MMP inhibitor, a mTor inhibitor, a NGFR tyrosine kinase inhibitor, a p38 MAP kinase inhibitor, a p56 tyrosine kinase inhibitor, a PDGF pathway inhibitor, a phosphatidylinositol-3-kinase inhibitor, a phosphatase inhibitor, a protein phosphatase inhibitor, a PKC inhibitor, a PKC delta kinase inhibitor, a polyamine synthesis inhibitor, a proteasome inhibitor, a PTP1B inhibitor, a SRC family tyrosine kinase inhibitor, a Syk tyrosine kinase inhibitor, a Janus (JAK-2 and/or JAK-3) tyrosine kinase inhibitor, a retinoid, a RNA polymerase II elongation inhibitor, a Serine/Threonine kinase inhibitor, a sterol biosynthesis inhibitor, a VEGF pathway inhibitor, an immunosuppressive agent, a CYP3A4 inhibitor, an anti-microbial agents, and an antiemetic, or a combination thereof.

39. The method of claim 38, wherein said MEK inhibitor is selected from the group consisting of butanedinitrile, GSK1120212, XL518, selumetinib, bis[amino[2-aminophenyl)thio]methylene]-(9Cl), (N-(3-chloro-4-fluorophenyl)-7-methoxy-6-(3-morpholin-4-ylpropoxy)quinazol-in-4-amine), (N-[(2R)-2,3-dihydroxypropoxy]-3,4-difluoro-2-[(2-fluoro-4-iodophenyl)amino]-benzamide), (2′-amino-3′-methoxyflavone), (1,4-diamino-2,3-dicyano-1,4-bis(aminophenylthio)butadiene), (6-(4-Bromo-2-chlorophenylamino)-7-fluoro-3-methyl-3H-benzoimidazole-5-carboxylic acid (2-hydroxyethoxy)-amide, [2-(2-fluoro-4-iodophenylamino)-N-(2-hydroxyethoxy)-1,5-dimethyl-6-oxo-1,6--dihydropyridine-3-carboxamide, (2-(2-Chloro-4-iodophenylamino)-N-cyclopropylmethoxy-3,4-difluoro-benzamide), N-[(R)-2,3-Dihydroxypropoxy]-3,4-difluoro-2-(2-fluoro-4-iodophenylamino)-benzamide, and U0126, or a combination thereof.
